(19) United States
(12) Patent Application Publication
(10) Pub. No.: US 2024/0009480 A1
(43) Pub. Date: Jan. 11, 2024

(54) WIRELESS BRAIN-COMPUTER INTERFACE

(71) Applicants: AARHUS UNIVERSITET, Aarhus C (DK); Region Midtjylland, Viborg (DK)

(72) Inventors: Farshad Moradi, Risskov (DK); Milad Zamani, Risskov (DK); Amin Rashidi, ZG Delft (NL); Jens Christian Hedemann Sørensen, Egå (DK)

(21) Appl. No.: 18/023,809

(22) PCT Filed: Sep. 1, 2021
(86) PCT No.: PCT/EP2021/074095

§ 371 Date: Feb. 28, 2023

(30) Foreign Application Priority Data
Sep. 1, 2020 (EP) 20193847.9

(51) Int. Cl. A61N 5/06; H02J 50/15; H02J 50/40

(52) U.S. Cl. CPC A61N 5/062; H02J 50/15; H02J 50/40; A61N 5/0622; A61N 5/0601; A61N 2005/0626; A61N 2005/0651

(57) ABSTRACT

A three-layer brain-computer interface system includes at least one external device, a plurality of second devices implanted on the surface of the cortex of a subject's brain and a plurality of micro devices implanted deeper within the brain. The external device provides power to the second devices and receives data from the second devices. The second devices include data and power receiving/transmission means and sensors, such as for electrocorticography, and provide power and data to the micro devices, such as ultrasound data and power transmission. The micro devices include sensor means and micro LEDS for measuring electric, chemical or other brain signals and provide brain stimulus through micro LEDs or other stimulating means, such as electric or chemical stimulation. The micro devices also receive and transmit data to and from the second devices. The system provides an energy efficient wireless measuring and stimulus system for implantation in the brain tissue.

### CROSS-REFERENCE TO RELATED APPLICATIONS

This application is the U.S. National Stage of PCT/EP2021/074095 filed on Sep. 1, 2021, which claims priority to European Patent Application 20193847.9 filed on Sep. 1, 2020, the entire content of both are incorporated herein by reference in their entirety.

### FIELD OF THE INVENTION

The present invention relates to a device and method for interfacing between a computer and brain tissue.

### BACKGROUND OF THE INVENTION

Within biomedical engineering there is a plurality of technologies for measuring and monitoring in vivo physiological properties within brain tissue either within a patient or an animal, such as biochemical sensors, electrocorticography (ECOG), magnetoencephalography (MEG) and electroencephalography (iEEG) for electrophysiological monitoring of brain activity.

One challenge, when attempting to measure one or more of the above is the short time period available for said measurements, as the setting for one or more measurements can primarily be performed in a clinical setting, due to power and data transmission, with a transcutaneous wire between the measuring device and an external device for data transmission and power supply to/from said measuring device. As some pathologies or physiological irregularities does not necessarily present acutely, within a short timeframe, the data from such a measurement is not always helpful to a physician or researcher.

For prolonged measurements periods, such as a week or several weeks, a wireless implant is a better solution but this presents other challenges relating to a sufficient power supply, as wireless implants with or without battery means needs a wireless power configuration to function for a prolonged time period. Furthermore, an implant without wireless data capabilities requires a memory in order to obtain the measured data. If the implant has a memory which is to be retrieved, the implant needs to be collected from the tissue after completing the measurement period, which implies further discomfort to a patient. If the implant has a wireless data transmission configuration, this renders more power consumption, which needs to be sufficiently supplied by the wireless power configuration. Radio frequency (RF) power, such as induction power, is a suitable method for supplying power to a wireless implant, but RF power has its limits relating to the tissue depth of the implant, in which tissue may be damaged from heating due to absorption of energy. Especially, within brain tissue, RF powering poses a problem, as the brain is sensitive to RF absorption and may harm the brain tissue or cause harmful physiological or neurological effects within a patient. Hence, if a wireless implant needs to be implanted within biological tissue, such as brain tissue, other wireless power technologies needs to be applied. Ultrasonic power transmission is an alternative method of wireless power transmission. Although the ultrasonic power transmission is high for tissues in comparison to RF, the range for ultrasonic power transmission is relatively low and the absorption from some tissues, such as bone e.g. the cranium is high and thus, ultrasonic power transmission between an external device and a wireless implant within the brain is not a viable solution for long term measurement of brain activity.

Furthermore, a range of chronic or intermittent pathologies is currently difficult or very invasive to treat, as current therapies or therapeutic implants face one or more of the above-mentioned challenges. Some of the limitations for therapeutic implants are size, i.e. too large for implantation, means of data transmission to/from said implant and, as stated above, sufficiently powering the device for a prolonged period of time. Furthermore, some of the therapies involved to be adapted for an implant may require specific integrated therapeutic means, which further increases the power usage of said implant.

Hence, an improved system or device for wireless interfacing to in vivo brain tissue and wireless therapy would be advantageous, and in particular, a more efficient and/or reliable wireless brain-computer interface (BCI) would be advantageous.

### OBJECT OF THE INVENTION

It is a further object of the present invention to provide an alternative to the prior art.

In particular, it may be seen as an object of the present invention to provide a wireless brain-computer interface and therapy system that solves the above mentioned problems of the prior art with providing power and two-way data transmission between a computing device and brain tissue, CSN tissue or spinal cord tissue, in vivo.

### SUMMARY OF THE INVENTION

Thus, the above described object and several other objects are intended to be obtained in a first aspect of the invention by providing a brain-computer interface system, the system comprising
a. —a first device for communicating with one or more implanted devices, the first device comprising
i.—wireless power transmission means,
ii.—wireless data communication means,
b. —a second device for implantation into tissue, such as brain tissue, CNS tissue or spinal cord tissue, the second device comprising
i.—wireless power receiving means for powering the second device, from the wireless power transmission means of the first device,
ii.—wireless data communication means for transmitting/receiving data to/from the wireless data communication means of the first device,
iii.—ultrasonic transducer means for transmitting and receiving ultrasound,
iv.—a controller configured for controlling at least the wireless data communication means and the ultrasonic transducer means of the second device,
c. —a micro device for implantation into tissue, such as brain tissue, CNS tissue or spinal cord tissue, the micro device comprising
i.—ultrasonic transducer means for receiving and/or transmitting ultrasound,
ii.—a power circuit for receiving power for powering the micro device by an ultrasonic signal received by the ultrasonic transducer of the micro device, from the ultrasonic transducer of the second device,
iii.—at least a first sensor configured for measuring signals from the tissue and to generate an analog electrical signal accordingly,
iv.—at least a first light source,
v.—a controller for controlling at least the first light source,
vi.—processing means for processing data to/from at least the ultrasonic transducer means, the at least first sensor and the controller, and
d. wherein the ultrasonic transducer of the micro device is further configured to receive data from the ultrasonic transducer of the second device.

In a preferred embodiment of the invention, the system is suitable for sending and receiving signals to and from in vivo tissue such as the brain tissue of a patient or subject. The first device may be positioned externally on the subjects head, one or more second devices may be implanted caudal to the first device, within the braincase, on one or more of the meninges or on or in the surface of the cortex of the brain and the one or more micro devices may be implanted deeper within the brain tissue, caudal to the first device and one or more second devices.

In the context of the present system an interface between tissue and a computer, is to be understood as the transfer of data and signals to and/or from a computer device and tissue, such as brain tissue, CNS tissue or spinal cord tissue. As an example, a brain-computer interface is a computer-based system that acquires brain signals, analyzes them, and translates them into commands that are relayed to an output device to carry out a desired action. In another example, a brain-computer interface is a computer-based system that sends signals to the brain, such as stimulating signals. In yet another example, As an example, a brain-computer interface is a computer-based system that sends/receives signals to/from the brain, such as for the purpose of treating an illness, based on received signals and wherein the system sends stimulus to the brain.

In an embodiment of the invention, the system is a closed-loop system which provides signals from the brain, central nervous system (CNS) or spinal cord to be processed by said system and wherein the system provides a response, such as an electric, chemical or other signal, to a part of the brain CNS or spinal cord, as a reaction or counteraction to said signals from the brain, CNS or spinal cord.

In another embodiment of the invention, the micro devices and/or second devices are implanted in or near the CNS) or the spinal cord.

In yet another embodiment of the invention, the micro devices and/or second devices are implanted in the brain and/or CNS and/or the spinal cord.

In yet another embodiment of the invention, measurement data is transferred from the brain, CNS or spinal cord to a computing device and stimulating signals are sent to the brain, CNS or spinal cord based on said measurement data.

In the context of the present invention, wireless power transmission is to be understood as power transmitted from a first device to one or more second devices without the use of a physical wire, e.g. power supplied via radiofrequencies, light or through ultrasound transduction.

In the context of the present invention, wireless data communication is to be understood as data transmitted between one or more first devices and one or more second devices without the use of a physical wire, e.g. light, such as infrared/optical data transfer, radio waves, magnetic data transfer, other electromagnetic data transfer or sound, such as ultrasound.

In the context of the present invention, ultrasonic transducer means is to be understood as a device that generate or sense energy from an ultrasonic signal and either converts an electric signal into ultrasound or converts ultrasound into an electric signal.

In an embodiment of the invention, an ultrasonic signal is generated from a first device and received from one or more second devices. The ultrasonic signal received from the one or more second devices can be used to receive data, such as instructions to said secondary devices and/or power said secondary devices.

In the context of the present invention, a controller is to be understood as a device or piece of equipment used to operate one or more peripheral devices, such as to operate or drive a secondary device based on instructions send from a first device as in the present invention.

In the context of the present invention, a micro device is to be understood as an electronic device manufactured using microfabrication, said micro device being suitable for insertion within the brain tissue, CNS tissue or spinal cord tissue with negligent damage to any surrounding tissue.

In an advantageous embodiment of the invention, the size of the micro device is small, and for implantation purposes, it may be preferred that the micro device is as small as possible. In preferred embodiment, the dimensions of the micro device is within 1×1×1 mm (height×length×width), such as within 500×500×500 μm, such as within 400×400×400 μm, such as within 300×300×300 μm, such as within 200×200×200 μm and in some embodiments it may be seen as most preferably to be within 100×100×100 μm. It is to be understood that the micro device may preferably be even smaller than 100×100×100 μm in case the actual manufacturing technologies chosen allows to.

In preferred embodiments, the micro device has a total volume of less than 2 mm³, preferably less than 1 mm³, preferably less than 0.7 mm³, such as less than 0.5 mm³.

In some embodiments, the micro device have non-uniform height, length and width. Especially, the height, length, and width dimensions may be such as 200×150×100 μm, or such as 150×150×100 μm, or such as the micro device having a height within 0.5-1.5 mm, a length of 0.5-1.0 mm, and a width of 0.3-0.7 mm.

In a preferred embodiment of the invention, the micro device is a neural dust for implantation within the brain tissue of a subject.

In the context of the present invention, a power circuit is to be understood as an assemblage of electronic elements or components configured to transfer or convert power within an electronic circuit, such as a power block or other type of power management unit (PMU), configured to provide a power output.

In yet another embodiment, one or more of the first device, second device and micro device further comprises an electric regulator circuit, configured to optimize the power usage of the circuit components within said first device, second device or micro device.

In the context of the present invention, a sensor is to be understood as a device configured for measuring one or more of a physical, chemical or electrical input, such as a chemical sensor, a molecular sensor, an impedance sensor, a fluorescence sensor, a temperature sensor, a voltage or electrical activity sensor, an optical sensor, an electrical probe, a vibration sensor, a biochemical sensor, an electrochemical sensor, a radiation sensor or a pressure sensor.

In an embodiment of the invention, the sensor is configured for measuring an electrical change within the brain, CNS or spinal cord of a subject and transmitting a signal based on said measurement to a second device.

In the context of the present invention, processing means is to be understood as a processing unit configured for processing data, such as data received within the micro device from the second device or data from the one or more sensors embedded within said micro device.

In the context of the present invention, light source is to be understood as any wavelength of light, which may provide the tissue surrounding said light source with a stimulus.

The invention is particularly, but not exclusively, advantageous for transmitting/receiving data and instructions between the first and second device(s) and the micro device(s).

In a preferred embodiment of the invention, the first device powers the second device which in turn powers the micro device. Through the first device, an operator can wirelessly send and receive data or instructions to the second device(s) and the micro device(s), enabling the operator to stimulate tissue and receive measurement data from the second device(s) and/or the micro device(s).

In an embodiment of the invention, the first device wirelessly powers a plurality of second devices which in turn powers a plurality of micro devices and the first device can transmit/receive data to/from the micro devices through the second devices.

In another embodiment, two or more second devices are arranged on a flexible membrane in a pattern, such as four equally distanced second devices arranged on a silicone sheet.

This embodiment is particularly advantageous for easy implantation onto the meninges or cortex so as to save time, reducing the risk of harm to a patient.

In an advantageous embodiment of the invention, the micro device further comprises at least a second light source.

In another embodiment, the light wavelength emitted by the first light source is different from the light wavelength emitted by the at least second light source.

This embodiment is particularly advantageous for providing additional stimulus to the surrounding tissue, such as brain, CNS or spinal cord tissue.

In yet another embodiment, the second device further comprises at least a first light source and/or at least a first sensor.

In another advantageous embodiment, the first device further comprises signal processing means and/or memory means.

This embodiment of the invention is particularly advantageous for generating internally generated automated response to data received from the second devices, which may provide a faster instruction prompting a stimulus from the second device or micro device.

In yet another advantageous embodiment of the invention, the second device further comprises signal processing means and/or sensor means, such as electrodes for sensing electrical signals from tissue.

In preferred embodiment, The first and second device have processing means, in which the second device receive data from the micro devices or sensors attached to the second device and processes said data after which the second device sends at least some of these data to the first device for further and/or stronger or more powerful processing. The processing means on the micro device, the second device, the first device and/or an external computing device may be either brain-inspired computing implemented using spiking neural networks or conventional Digital Signal Processors (DSPs).

In an advantageous embodiment, the sensor means of the second device may comprise a Nano-wire arranged for measuring a Local Field Potential. The Nano-wire may furthermore be used to perform single cell recording.

This embodiment of the invention is particularly advantageous for sensing an input from the tissue, such as an electrical signal, and providing a corresponding stimulus to said tissue, such as an electrical stimulus.

In a preferred embodiment of the invention, the micro device further comprises stimulation means configured for stimulating tissue, e.g. electrical stimulus, chemical stimulus, pressure stimulus, temperature stimulus, vibrating stimulus, radiation stimulus or optical stimulus, said stimulation means being controlled by the controller.

This embodiment of the invention is particularly advantageous for providing a device suitable for treating an illness within the tissue, such as by stimulating the brain, CNS or spinal cord at a specific location, thus relieving a subject of a neurological ailment.

In another preferred embodiment of the invention, the ultrasonic transducer of the micro device is configured to transmit data from the processing means to the ultrasonic transducer of the second device.

In yet another preferred embodiment of the invention, the power circuit of the micro device further comprises an electric load modulation circuit configured to use ultrasonic backscattering as a means of transmitting data from the micro device to the ultrasonic transducer of the second device.

This embodiment is particularly advantageous for continuously or intermittently powering the micro device, by combining wireless power transfer between the second device and the micro device with wireless data transfer. By providing wireless power to the micro device, the invention is advantageous for long-term use, such as for long-term monitoring of the tissue of a patient and/or treatment by stimulus provided to the tissue from the micro device.

In another preferred embodiment of the invention, the first or second light source is configured to provide stimulus/therapy to the tissue, such as optogenetic or photodynamic stimulus/therapy within the brain, CNS or spinal cord of a subject.

In the context of the present invention, optogenetics is to be understood as a method that involves the use of light to control tissue, such as CNS cells or such as neurons, that have been genetically modified to express light-sensitive ion channels. As such, optogenetics is a neuromodulation method that uses a combination of techniques from optics and genetics to control the activities of individual neurons in tissue, such as brain tissue and may be suitable for providing vision support to a vision-impaired patient.

In the context of the present invention, photodynamic therapy is to be understood as phototherapy involving light and a photosensitizing chemical substance, such as for cancer therapy.

In an advantageous embodiment of the invention, the first or second light source is configured for providing controlled release of a drug, such as for providing a light controlled nanoparticulate drug delivery system.

In an embodiment of the invention, the micro device comprises a combination of optogenetic stimulation means and a light controlled drug delivery system.

In yet another embodiment, the micro device comprises a combination of optogenetic stimulation means, a light controlled drug delivery system and a third stimulation means, such as electrical or chemical stimulation means.

In another advantageous embodiment, the drug is controllably released from an implanted container by means of light applied to said implanted container from the first or second light source, said controlled drug release being based on the data received by the ultrasonic transducer of the micro device from the ultrasonic transducer of the second device.

In yet another advantageous embodiment, the drug is attached to the micro device before implantation, controllably released from the surface or an attached vessel integrated on or in the micro device by means of light applied to said attached drug from the first or second light source, said controlled drug release being based on the data received by the ultrasonic transducer of the micro device from the ultrasonic transducer of the second device. The attached drug may also be a coating or surface treatment, encapsulating at least a portion of the micro device.

This embodiment is particularly advantageous for precision medicine and/or targeted therapy.

In the context of the present invention, Targeted therapy is to be understood as a treatment that uses drugs to target specific genes and proteins that are involved in the growth and survival of malignant cells, such as cancer cells. Targeted therapy can affect the tissue environment that helps a malignant cell, such as cancer cell growth and survive or it can target cells related to cancer growth, such as blood vessel cells.

In a preferred embodiment of the invention, the wireless data communication means of the first and second device comprises optical or RF inductive data communication means.

In another preferred embodiment of the invention, the wireless power transmission means of the first device comprises RF induction power means.

In yet another preferred embodiment of the invention, the at least first sensor of the micro device comprises a Local Field Potential and/or an Action Potential sensor. The micro device may further comprise a plurality of secondary sensors. The Local Field Potential sensor may be a nanowire extending from the micro device and may further be used for single cell recording.

In an advantageous embodiment of the invention, the micro device further comprises a temperature sensor and/or a pressure sensor and/or a biochemical sensor and/or other suitable sensor relevant to the tissue in which the micro device is located.

This embodiment of the invention is particularly advantageous for providing an array of measurement date from the tissue in which said micro device is implanted.

In another preferred embodiment, the micro device further comprises memory means and wherein the micro device is configured to accumulate between 10 microseconds and 5.0 seconds of data, more preferably between 50 microseconds and 1.0 seconds of data or most preferably between 100 microseconds and 0.5 seconds of data from the one or more sensor means, before transmitting said data to the second device.

This embodiment of the invention is particularly advantageous for compiling data packages and transmitting said data packages to the second device, which in turn may process the data or transmit the data to the first device for further processing.

In an advantageous embodiment, the system further comprises a plurality of micro devices, said plurality of micro devices configured so as to transmit data to the second device at off-set intervals.

This embodiment is particularly advantageous for increasing the signal to noise ratio, ensuring a higher quality of the wireless data transmission and may furthermore be advantageous for identifying the position of a communicating micro device.

In another preferred embodiment, the system further comprises a plurality of implanted second devices, each of the implanted devices configured to provide power and send/receive data between said implanted device and an array of micro devices.

This embodiment of the invention is particularly advantageous for providing increased wireless data and power transmission speed, amount, and quality between the second devices and the micro devices.

In an embodiment of the invention, the system comprises a first device, which may be referred to as a host device, providing power and transmitting/receiving data to 20 second devices, each of said second devices being referred to as hubs, said 20 second devices further providing power and transmitting/receiving data to 20 micro devices, such as neural dusts, thus enabling data transmission between a first device and 400 micro devices, and hence providing communication between the biological tissue in which the second devices and the micro devices are implanted. It is to be understood that the system may comprise at least a first device, a second device and a micro device or at least 10 micro devices, at least 20 micro devices, at least 30 micro devices, at least 40 micro devices, at least 50 micro devices at least 100 micro devices or at least 500 micro devices.

In yet another preferred embodiment, the second devices are configured to transmit and receive data from other second devices, i.e. implanted hubs communicating with other implanted hubs and furthermore the micro devices may be configured to transmit and receive data from other micro devices, i.e. implanted dusts communicating with other implanted dusts.

In an advantageous embodiment, the processing means in any of the first device, the second device or the micro device is a spike-based neural network (SNN).

In the context of the present invention, a spike based neural network is to be understood as a spiking neural network in which data is converted to spikes and where time is encoded in the spike-encoded data. Some Neuromorphic Computing Systems (NCSs) use SNNs for processing data, which minimizes the volume of collected data. Furthermore, if the SNNs are configured in a closed-loop system to generate an electrical spike, said system has similarities to the function of the brain, which may lead to a more efficient stimulation, such as neuromodulation.

This embodiment of the invention is particularly advantageous for processing measured signals and communicating said signals in a less power and data transmission consuming way and furthermore enables the system to learn and adapt, based on previous inputs. Furthermore, SNN may provide faster response to a measured signal, such as a signal from a sensor means, immediately within a device, which may render an indication, treatment or therapy to be provided faster, e.g. detection of a seizure occurrence and generation of a stimulation signal, such as a counteracting, prophylactic or remedial stimulating signal.

In a second aspect, the invention relates to a method of measuring and/or sending signals from/to tissue, such as brain tissue, CNS tissue or spinal cord tissue, the method comprising:
a. —providing a first device for communicating with one or more implanted devices, the first device comprising
i.—wireless power transmission means,
ii.—wireless data communication means,
b. —providing a second device for implantation into tissue, the second device comprising
i.—wireless power receiving means for powering the second device, from the wireless power transmission means of the first device,
ii.—wireless data communication means for transmitting/receiving data to/from the wireless data communication means of the first device,
iii.—ultrasonic transducer means for transmitting and receiving ultrasound,
iv.—a controller configured for controlling at least the wireless data communication means and the ultrasonic transducer means of the second device,
c. —providing a micro device for implantation into tissue, the micro device comprising
i.—ultrasonic transducer means for receiving and/or transmitting ultrasound,
ii.—a power circuit for receiving power for powering the micro device by an ultrasonic signal received by the ultrasonic transducer of the micro device, from the ultrasonic transducer of the second device,
iii.—at least a first sensor configured for measuring signals from the tissue and to generate an analog electrical signal accordingly,
iv.—at least a first light source,
v.—a controller for controlling at least the first light source,
vi.—processing means for processing data to/from the ultrasonic transducer means, the at least first sensor and the controller, and
d. wherein the method further comprises to receive and/or send data from/to the ultrasonic transducer of the second device.

This aspect of the invention is particularly but not exclusively advantageous for providing wireless signals, such as data or power transfer, between a first device and a second device and a micro device, such as a neural dust. This embodiment of the invention may be used to provide long-term measurement data and/or treatments or therapies aimed towards a range of pathologies within a subject, such as, but not limited to an Alzheimer's patient.

In a third aspect, the invention relates to the use of the micro device or system according to the first aspect. Especially, use of the micro device or system for treatment or therapy on a living person or animal. Especially, the micro device is a so-called neural dust arranged for implantation into tissue, such as brain tissue, CNS tissue or spinal cord tissue, and being arranged for treatment or therapy of one or more diseases and/or pain. The micro device may be capable of single or double wavelength optical therapy or optogenetics for neuromodulation, and this may be combined with electric stimulation of the brain tissue to provide electric neuromodulation. In embodiment where the micro devices comprise an electric neural sensor, a closed loop control of the applied treatment or therapy may be provided.

In a fourth aspect, the invention relates to the treatment of a patient, using the method according the second aspect of the invention.

This aspect of the invention may be particularly advantageous for treating illnesses or pathologies such as, but not limited to chronic pain, depression, movement disorders, Parkinson's disease, Alzheimer's disease, epilepsy, blindness. The invention, as it revolves around measuring and providing signals and stimulus to and from brain, CNS or spinal cord tissue, may be suitable for treating a plurality of ailments relating to chemical, hormonal or electrical imbalances and may furthermore be used to transmit sensory or motor signals from the peripheral nervous system (somatic and autonomous system), which are not sufficiently transferred to the central nervous system, either due to trauma, prenatal diseases or other diseases related to the nervous system.

The first, second, third and fourth aspect of the present invention may each be combined with any of the other aspects. These and other aspects of the invention will be apparent from and elucidated with reference to the embodiments described hereinafter.

### BRIEF DESCRIPTION OF THE FIGURES

The brain-computer interface system, according to the invention will now be described in more detail with regard to the accompanying figures. The figures show one way of implementing the present invention and is not to be construed as being limiting to other possible embodiments falling within the scope of the attached claim set.

FIG. 1 is a cross-sectional view of the system applied to the head and brain of a subject, according to an embodiment of the invention;

FIG. 2 is an illustration of the system according to an embodiment of the invention, with an enlarged illustration of the first device;

FIG. 3 is an illustration of the system according to an embodiment of the invention, with an enlarged illustration of the second device;

FIG. 4 is an illustration of the system according to an embodiment of the invention, with an enlarged illustration of the micro device;

FIG. 5a and FIG. 5b illustrate a micro device embodiment and a system embodiment;

FIG. 6 is a flow-chart of a method according to the invention;

FIG. 7 shows a schematic diagram of an experimental setup of a brain-computer interface system, according to an embodiment of the invention;

FIG. 8 shows three graphs, representing transient measurement results from the experimental set up as shown in FIG. 7; and

FIG. 9 shows three graphs, representing measured acoustic intensity at the piezoelectric receiver, from the experimental set up as shown in FIG. 7.

### DETAILED DESCRIPTION OF AN EMBODIMENT

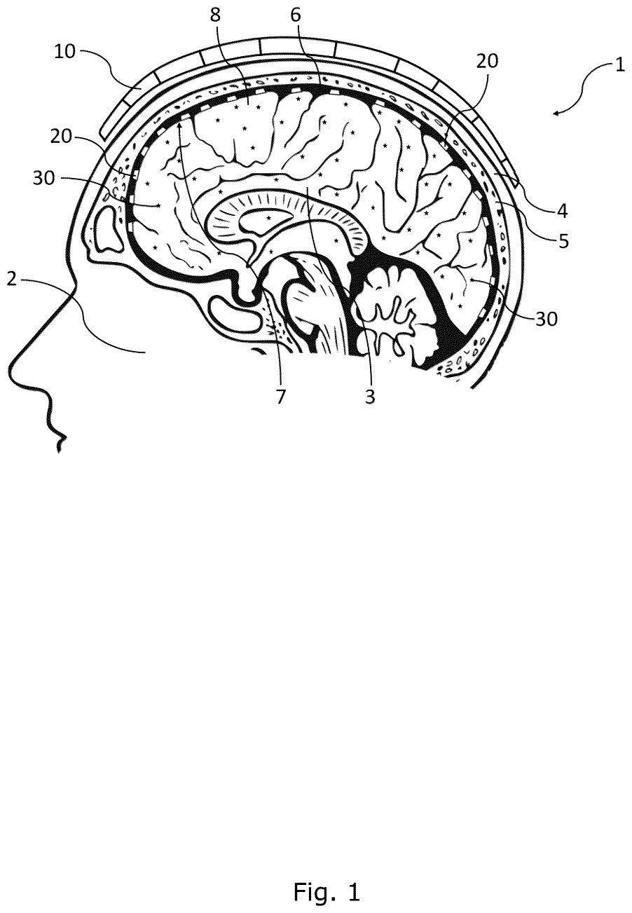

FIG. 1 is a cross-sectional view of the system 1 applied to the head 2 and brain 3 of a subject, according to an embodiment of the invention. FIG. 1 illustrates the relevant layers of the subjects head, with an outer layer of skin 4. Below the skin 4, the cranial bone 5 covers a membrane 6. Below the membrane 6, the cortex 8 of the brain 3 is situated along the peripheral circumference of the brain 3. The system 1 is illustrated by three layers of devices in which the outer device, on the outside of the skin 4, is the first device 10 or the host device. Within the head 2 of the subject, a plurality of second devices 20 or hubs, are implanted along the surface 7 of the cortex 8, below or within the membrane 6. Deeper within the brain 3, a plurality of micro devices 30 or neural dusts are implanted. It should be noted that the system 1 is not to scale, relative to the subjects head 2.

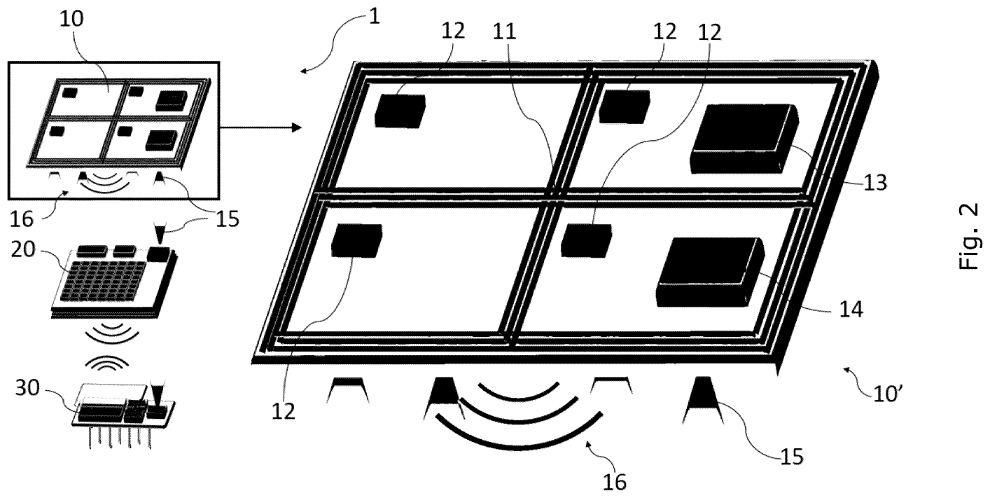

FIG. 2 is an illustration of the system 1, according to an embodiment of the invention. FIG. 2 illustrates the setup between the first device 10 and the second device 20 and between the second device 20 and the micro device 30, wherein the first device 10 transmits power 16 to the second device 20 and use optical data communication 15 to communicate with the second device 20. Furthermore FIG. 2 shows an enlarged illustration of the first device 10′, showing a circuit 11 with four LED's 12, a microchip or application-specific integrated circuit 13, a memory 14, optical data communication 15 and wireless power transmission 16.

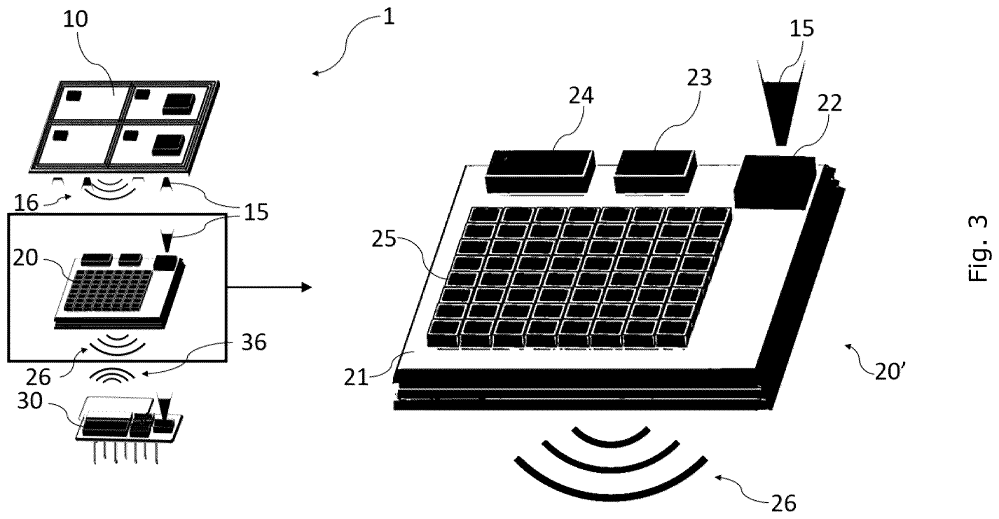

FIG. 3 is an illustration of the system according to an embodiment of the invention, with an enlarged illustration of the second device. FIG. 3 illustrates the setup between the first device 10 and the second device 20 and between the second device 20 and the micro device 30, wherein the second device 20 receives power 16 from the first device 10, use optical data communication 15 to communicate with the first device 10, transmits power and data 26 to the micro device 30 and receives data 36 from the micro device 30. Furthermore FIG. 3 shows an enlarged illustration of the second device 20′, showing a circuit 21 with an LED 22, a microchip or application-specific integrated circuit 23, a memory 24, optical data communication 15 and wireless power transmission 26 provided by a piezoelectric circuit or transducer 25.

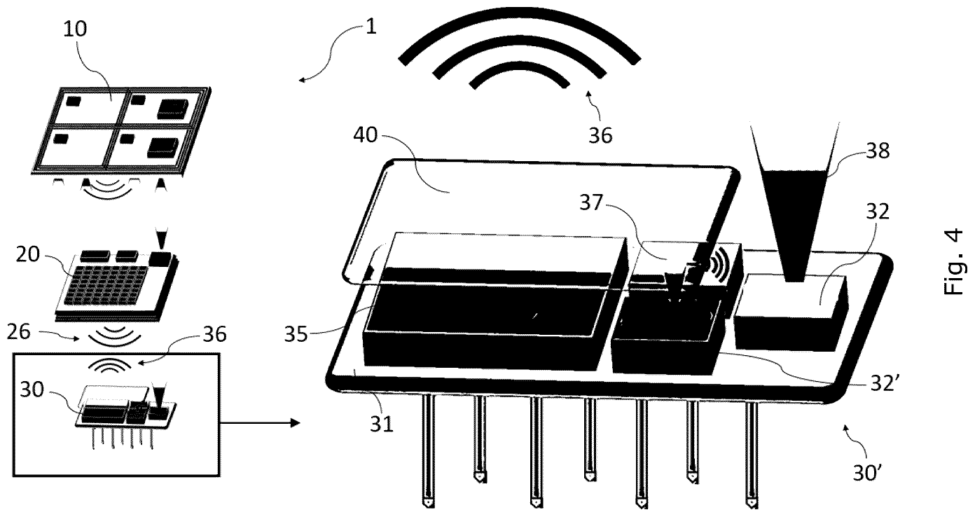

FIG. 4 is an illustration of the system according to an embodiment of the invention, with an enlarged illustration of the micro device. FIG. 4 illustrates the setup between the first device 10 and the second device 20 and between the second device 20 and the micro device 30, wherein the micro device 30 receives power 26 from the second device 20 and transmits and receives data 36 to and from the second device 20. Furthermore FIG. 4 shows an enlarged illustration of the micro device 30′, showing a circuit 31 with a micro LED 32 for controlling drug release by applying light to an implanted drug container 40, a second micro LED 32 for therapy of brain tissue, an integrated circuit 37 and a piezoelectric circuit or transducer 35 for receiving power and receiving and transmitting data 36 to and from the second device 20.

In a preferred embodiment of the invention, the second micro LED 32 is configured for optogenetic therapy of brain tissue.

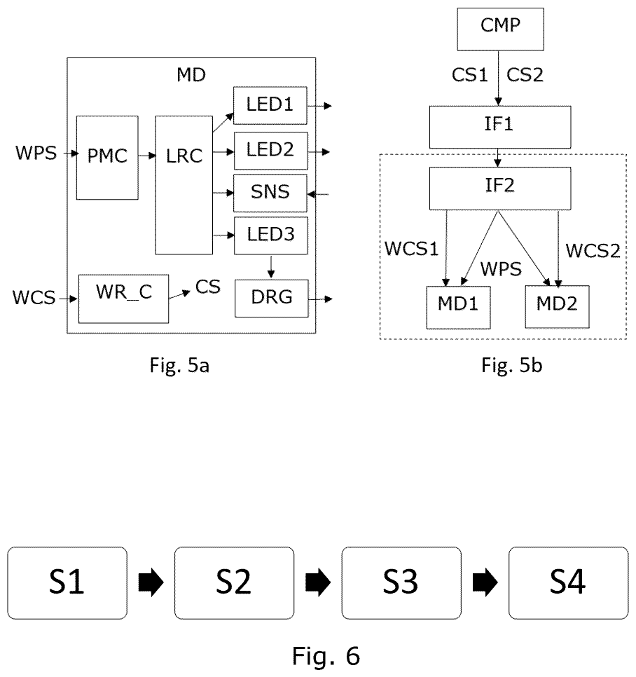

FIG. 5a illustrates a micro device MD, e.g. a neural dust, embodiment which receives a wireless power signal WPS, preferably an ultrasonic signal, and a wireless control signal WCS, may be an ultrasonic and/or an electromagnetic RF signal. A power management circuit PMC receives the wireless power signal WPS and generates a power output accordingly for powering all power consuming components of the dust, here including three light sources LED1, LED2, LED3, and a sensor SNS. A wireless receiver WR_C receives the wireless control signal WCS and provides a control signal CS accordingly, for control of the light sources LED1, LED2, LED3. Preferably, two light sources LED1, LED2 can generate optogenetics and/or optical therapy at different light wavelengths, while one light source LED3 can generate light for optically triggering drug deliver to surrounding biological tissue by providing light on a drug container DRG inside the dust.

FIG. 5b illustrates a system embodiment with a three layer approached for communication and powering of two implantable brain dusts MD1, MD2. A computer CMP outside a person's body communicates control signals CS1, CS2 for controlling function of the respective brain dusts MD1, MD2. The computer is connected to a first interface part IF1 to be placed on the head of a person, i.e. outside the skull. This first interface part IF1 communicates wirelessly with a second interface part IF2 which is arranged for implantation inside the skull of the person. This second interface part IF2 serves to provide power to the brain dusts MD1, MD2 by transmitting ultrasonic power signals WPS through the brain tissue to the implanted dusts MD1, MD2. Further, wireless control signals WCS1, WCS2 to the respective dusts MD1, MD2 are also transmitted, e.g. via ultrasonic signal or via electromagnetic RF signals. In this way, a computer CMP to brain interface can be implemented, and various functions of the dusts MD1, MD2, as the example device shown in FIG. 5a, can be individually controlled, e.g. to provide an electrical, drug and/or optical treatment, therapy, and/or to monitor neural activity.

FIG. 6 is a flow-chart of a method according to the invention, for measuring and/or sending signals from/to tissue such as brain tissue, CNS tissue or spinal cord tissue, the method comprising the following steps:
a. S1—providing a first device for communicating with one or more implanted devices, the first device comprising
i.—wireless power transmission means,
ii.—wireless data communication means,
b. S2—providing a second device for implantation into tissue, the second device comprising
i.—wireless power receiving means for powering the second device, from the wireless power transmission means of the first device,
ii.—wireless data communication means for transmitting/receiving data to/from the wireless data communication means of the first device,
iii.—ultrasonic transducer means for transmitting and receiving ultrasound,
iv.—a controller configured for controlling at least the wireless data communication means and the ultrasonic transducer means of the second device,
c. S3—providing a micro device for implantation into tissue, the micro device comprising
i.—ultrasonic transducer means for receiving and/or transmitting ultrasound,
ii.—a power circuit for receiving power for powering the micro device by an ultrasonic signal received by the ultrasonic transducer of the micro device, from the ultrasonic transducer of the second device,
iii.—at least a first sensor configured for measuring signals from the tissue and to generate an analog electrical signal accordingly,
iv.—at least a first light source,
v.—a controller for controlling at least the first light source,
vi.—processing means for processing data to/from the ultrasonic transducer means, the at least first sensor and the controller, and

S4 wherein the method further comprises to receive and/or send data from/to the ultrasonic transducer of the second device.

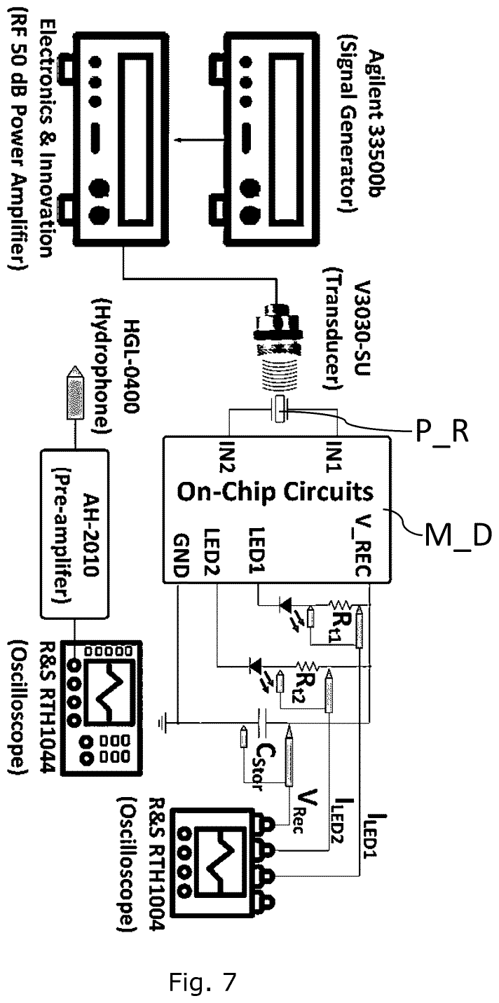

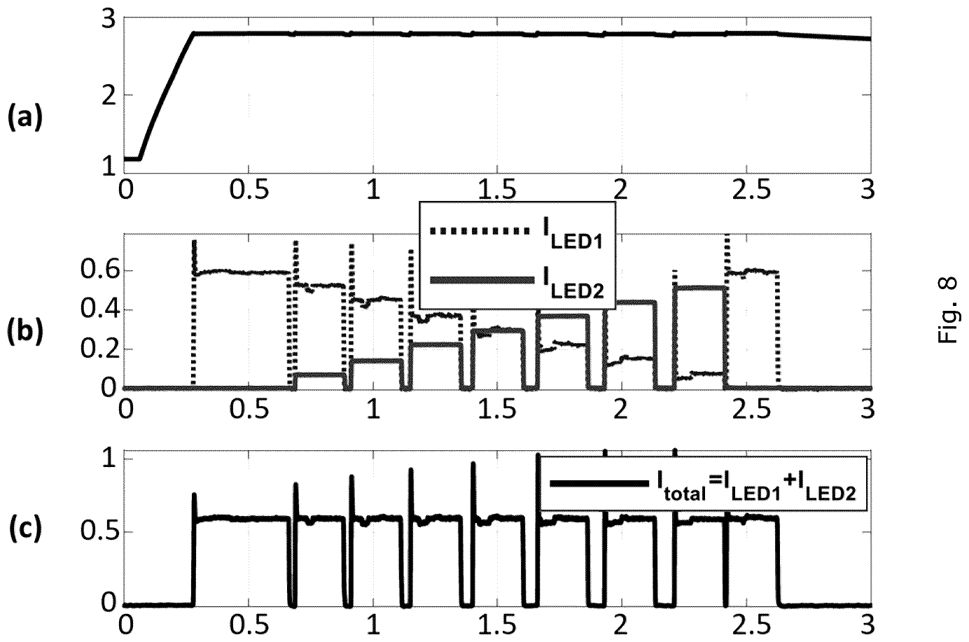

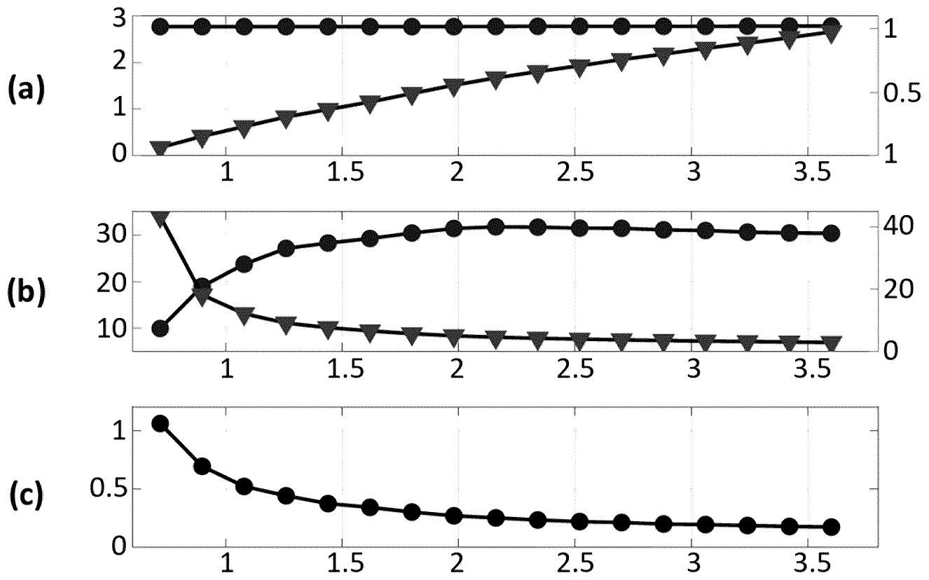

FIG. 7 shows a schematic diagram of an experimental setup of a brain-computer interface system, according to an embodiment of the invention. The inventors have set up a live experimental prototype, according to an embodiment of the invention, for the purpose of measuring dual-wavelength light, i.e. optogenetic signals, when the micro device M_D is powered by ultrasonic waves. The schematic diagram shows how the set up was built and how the experiment was performed. A 2.55 ms ultrasonic burst, including a series of duration-increasing notches is fed into an arbitrary signal generator, Agilent 33500b, and transmitted, as an ultrasonic power burst to the piezoelectric receiver P_R on the micro device M_D, through an amplifier, RF 50 dB power amplifier, and a transducer, V3030-SU. The transducer successfully powers two LED's LED1, LED2 and the light emitted from the LED's LED1, LED2 was measured, represented by two connected oscilloscopes, R&S RTH 1044, which was proved by the measurements provided in graphs in FIG. 8 and FIG. 9 respectively. A hydrophone was connected to the system to verify the signal from the transducer.

FIG. 8 shows three graphs representing transient measurement results from the experimental set up as shown in FIG. 7.

The upper graph (a) shows Vrec at the micro devices M_D output, with the y-axis representing voltage and the x-axis representing time in milliseconds.

The middle graph (b) shows electric current of I_{LED1}, dotted line and I_{LED2}, solid line, at the micro devices M_D output, with the y-axis representing current in milliamps and the x-axis representing time in milliseconds.

The lower graph (c) shows total load current for I_{LED1} and I_{LED2}, at the micro devices M_D output, with the y-axis representing current in milliamps and the x-axis representing time in milliseconds.

As shown in (b), I_{LED2} increases stepwise from 0 to 514 μA with steps of 74 μA +/−5%, according to the encoded commands over the notch durations, while I_{LED1} takes the rest of the current budget. Thus, VRec, and I_{Total}=I_{LED1}+I_{LED2} are regulated to 2.79V, and 600 μA, respectively.

FIG. 9 shows three graphs (a), (b), (c) representing measured acoustic intensity at the piezoelectric receiver (in mW/mm², x-axis) P_R, from the experimental set up as shown in FIG. 7. In these measurements, time-average intensity of 2.5 ms ultrasonic power bursts are swept from 0.72 to 3.6 mW/mm² with steps of 0.18 mW/mm². The voltage Vrec and DC current through LED1, and startup time have been measured. The upper graph (a) shows Vrec voltage (in V indicated on the left y-axis, shown with circles) and corresponding DC current (in mA indicated on the right y-axis, shown with triangles). As the acoustic intensity increases, current through LED1 increases while Vrec stays regulated to 2.79 V ±0.5%. Thus the DC resistance decreases. The middle graph (b) shows the efficienty (electrical power at LED1 divided by the acoustic power at the piezo surface) shown with circles in % to the left, and the DC electrical load is shown with triangles (in kΩ) to the right. The lower graph (c) shows that startup time (in ms) of the chip reduces non-linearly by increasing the acoustic power.

To sum up, the invention provides a three-layer brain-computer interface system comprising at least one external device, a plurality of second devices implanted on the surface of the cortex of a subjects brain and a plurality of micro devices implanted deeper within the brain. The external device is configured to provide power to the at least the plurality of second devices, such as by induction power and transmit and receive data from at least the plurality of second devices. The second devices comprises data and power receiving/transmission means and sensors, such as for electrocorticography, and are configured for providing power and data to the plurality of micro devices, such as ultrasound data and power transmission. The micro devices comprises sensor means and micro LEDS for measuring electric, chemical or other signals from the brain and are configured to provide stimulus to the brain through the micro LED's or other stimulating means, such as electric or chemical stimulation. The micro devices are further configured to receive and transmit data to and from the second devices. The system provides an energy efficient wireless measuring and stimulus system for implantation in the brain tissue.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1.-16. (canceled)

17. A brain-computer interface system, the system comprising
a first device for communicating with one or more implanted devices, the first device comprising;
a wireless power transmitter; and
a wireless data communication unit;
a second device for implantation into tissue, such as brain tissue, central nervous system (CNS) tissue or spinal cord tissue, the second device comprising;
a wireless power receiver for powering the second device, from the wireless power transmitter of the first device;
a wireless data communication unit for transmitting/receiving data to/from the wireless data communication unit of the first device;
an ultrasonic transducer for transmitting and receiving ultrasound; and
a controller configured for controlling at least the wireless data communication unit and the ultrasonic transducer of the second device;
a micro device for implantation into tissue, such as brain tissue, CNS tissue or spinal cord tissue, the micro device comprising;
an ultrasonic transducer for receiving and/or transmitting ultrasound;
a power circuit for receiving power for powering the micro device by an ultrasonic signal received by the ultrasonic transducer of the micro device, from the ultrasonic transducer of the second device;
at least a first sensor configured for measuring signals from the tissue and to generate an analog electrical signal accordingly;
at least a first light source;
a controller for controlling at least the first light source; and
a processing unit for processing data to/from at least the ultrasonic transducer, the at least first sensor and the controller; and
wherein the ultrasonic transducer of the micro device is further configured to receive data from the ultrasonic transducer of the second device.

18. The system according to claim 17, wherein the micro device further comprises at least a second light source.

19. The system according to claim 17, wherein the light wavelength emitted by the first light source is different from the light wavelength emitted by the at least second light source.

20. The system according to claim 17, the first and/or second device further comprising a signal processing unit and/or a memory.

21. The system according to claim 17, the second device further comprising one or more sensors, such as electrodes for sensing electrical signals from tissue.

22. The system according to claim 17, the micro device further comprising stimulation means configured for stimulating tissue, said stimulation means being controlled by the controller.

23. The system according to claim 17, wherein the ultrasonic transducer of the micro device is configured to transmit data from the processing unit to the ultrasonic transducer of the second device.

24. The system according to claim 18, wherein the first or second light source is configured to provide stimulus/therapy to tissue, such as optogenetic or photodynamic stimulus/therapy.

25. The system according to claim 18, wherein the first or second light source is configured for providing controlled release of a drug.

26. The system according to claim 17, wherein at least a first sensor of the micro device comprises a Local Field Potential and/or an Action Potential sensor.

27. The system according to claim 17, wherein the micro device further comprises a temperature sensor and/or a pressure sensor and/or a biochemical sensor.

28. The system according to claim 17, the micro device further comprising a memory and wherein the micro device is configured to accumulate a time period T of data from the one or more sensors, before transmitting said data to the second device.

29. The system according to claim 17, further comprising a plurality of micro devices, said plurality of micro devices configured so as to transmit data to the second device at off-set intervals.

30. The system according to claim 17, wherein the processing unit in any of the first device, the second device or the micro device comprises a spike-based neural network.

31. The system according to claim 17, wherein the micro device has a total volume of less than 1 mm³, such as less than 0.5 mm³, such as less than 0.2 mm³.

32. A method of measuring and/or sending signals from/to tissue, such as brain tissue, CNS tissue or spinal cord tissue, the method comprising:
providing a first device for communicating with one or more implanted devices, the first device comprising;
a wireless power transmitter; and
a wireless data communication unit;
providing a second device for implantation into tissue, the second device comprising;
a wireless power receiver for powering the second device, from the wireless power transmitter of the first device;
a wireless data communication unit for transmitting/receiving data to/from the wireless data communication unit of the first device;
an ultrasonic transducer for transmitting and receiving ultrasound;
a controller configured for controlling at least the wireless data communication unit and the ultrasonic transducer of the second device;
providing a micro device for implantation into tissue, the micro device comprising;
an ultrasonic transducer for receiving and/or transmitting ultrasound;
a power circuit for receiving power for powering the micro device by an ultrasonic signal received by the ultrasonic transducer of the micro device, from the ultrasonic transducer of the second device;
at least a first sensor configured for measuring signals from the tissue and to generate an analog electrical signal accordingly;
at least a first light source;
a controller for controlling at least the first light source;
a processing unit for processing data to/from the ultrasonic transducer, the at least first sensor and the controller; and
wherein the method further comprises to receive and/or send data from/to the ultrasonic transducer of the second device.
